(19) United States
(12) Patent Application Publication
(10) Pub. No.: US 2005/0214175 A1
(43) Pub. Date: Sep. 29, 2005

(54) Lunimair airfresh comined unit

(76) Inventor: Derrick John Martin Barker, Worcestershire (GB)

Correspondence Address: EDWARDS & ANGELL, LLP, P.O. BOX 55874, BOSTON, MA, 02205, US

(21) Appl. No.: 10/514,678

(22) PCT Filed: May 14, 2003
(86) PCT No.: PCT/GB03/02064

§ 371 Date: Nov. 15, 2004

(30) Foreign Application Priority Data
May 15, 2002 (GB) 0211095.5
May 2, 2003 (GB) 0310123.5

(51) Int. Cl. A61L 9/03

(52) U.S. Cl. 422/124; 422/125

(57) ABSTRACT

The invention relates to a ceiling mounted fragrance distributing device or airfreshener that can be battery or mains powered. The device incorporates a cyclonic airflow motion achieved by directional inlets and radial walls within the body of the device. As an extension of the device a light source is included providing a combined and airfreshener device.

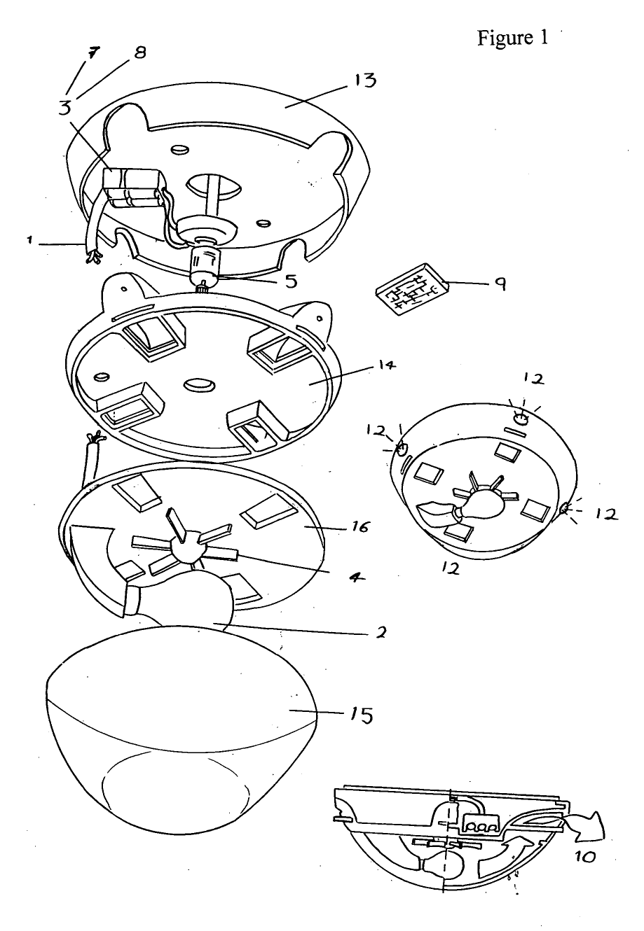

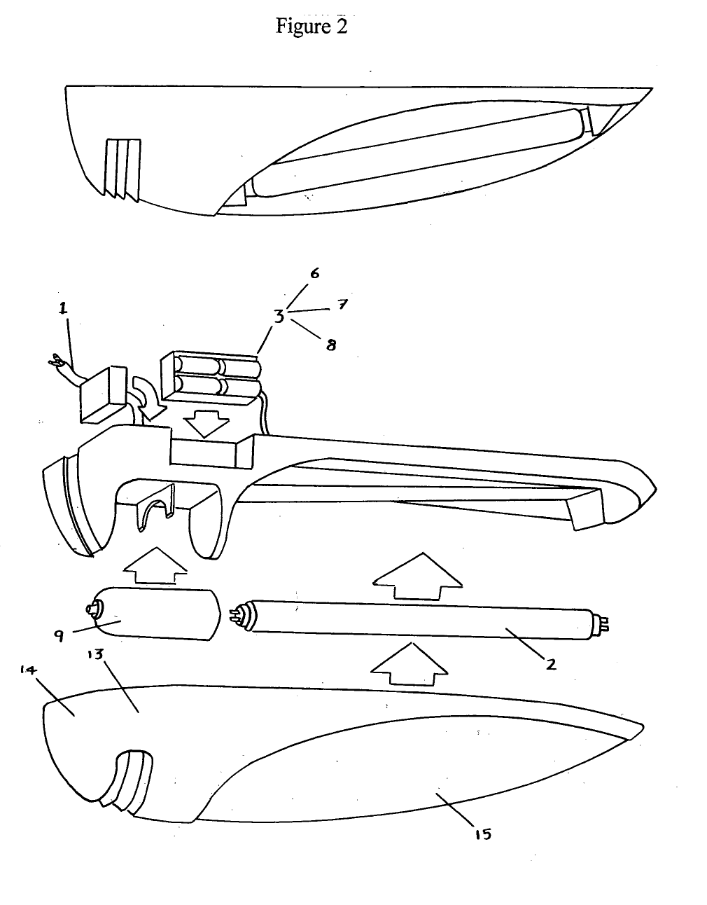

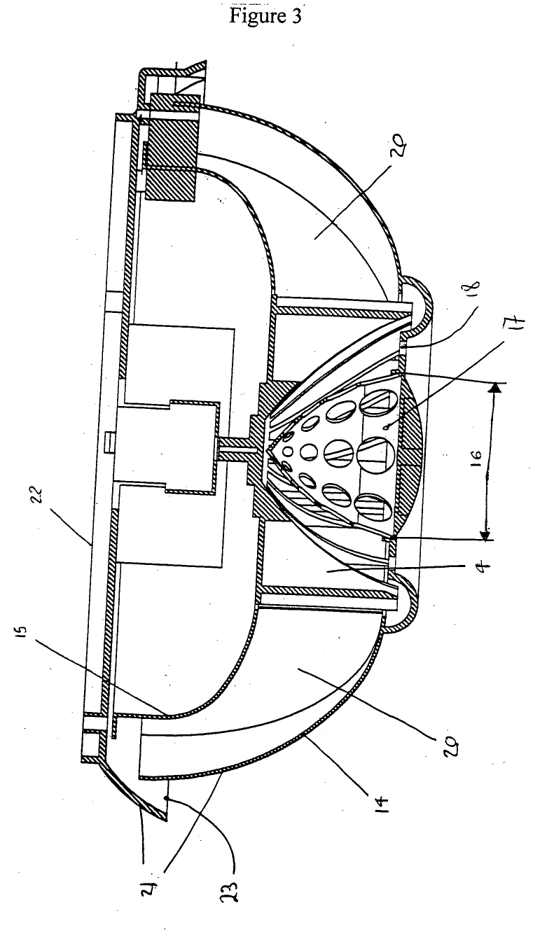

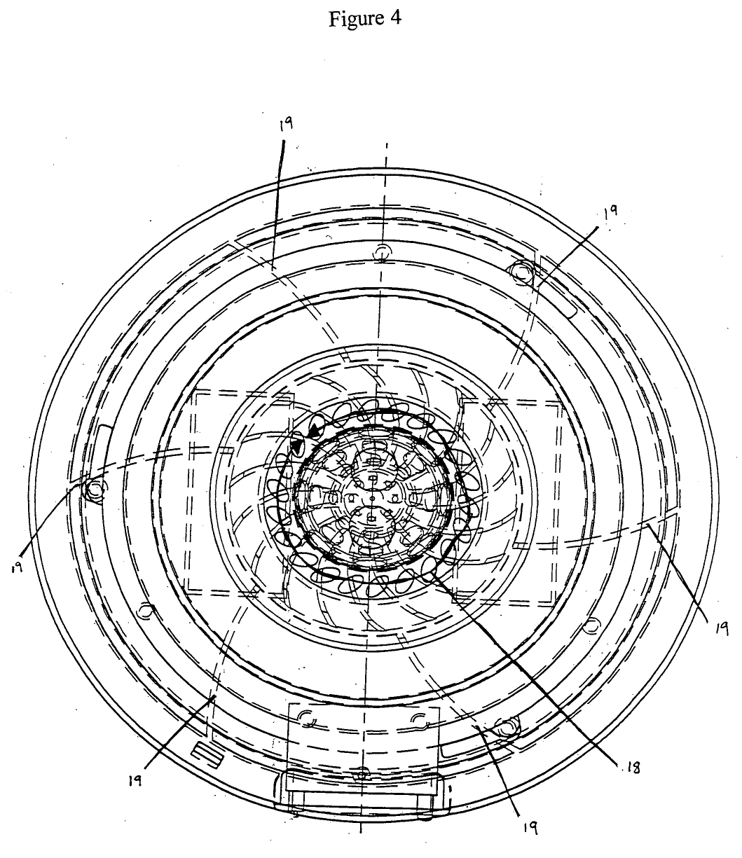

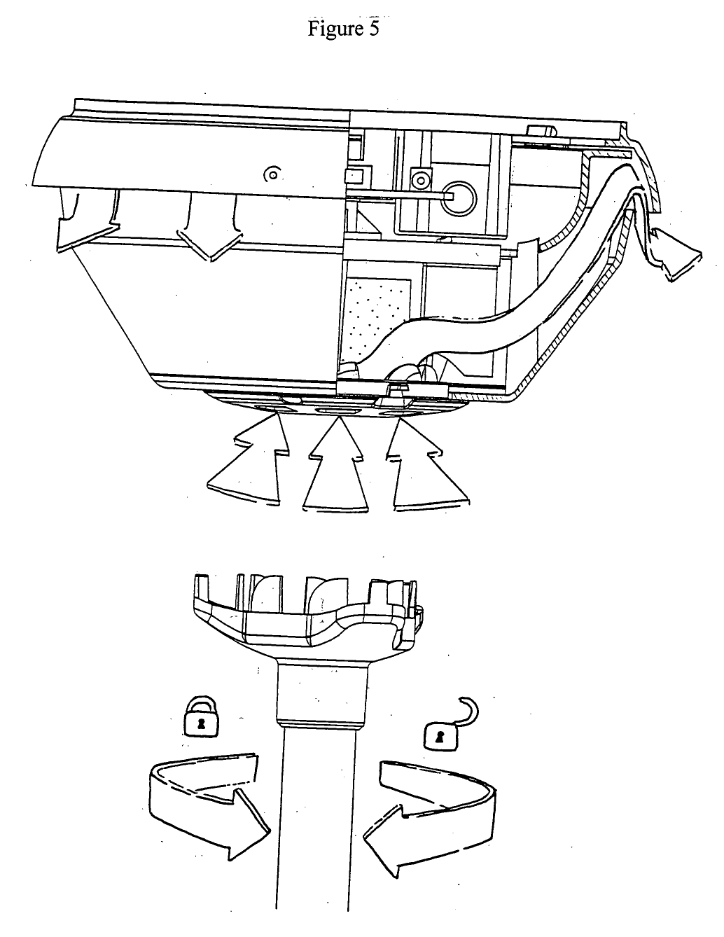

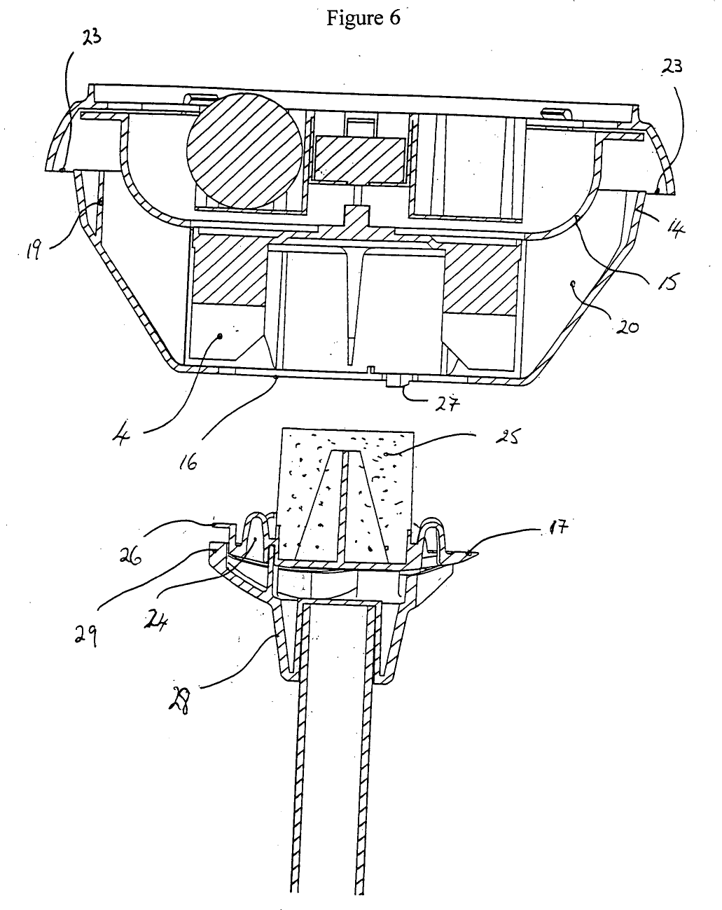

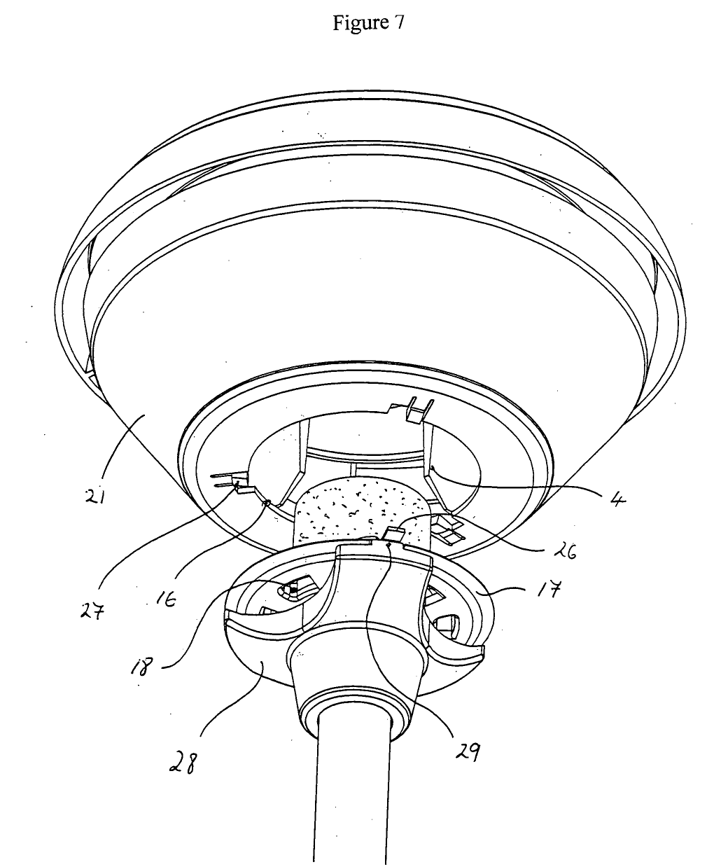

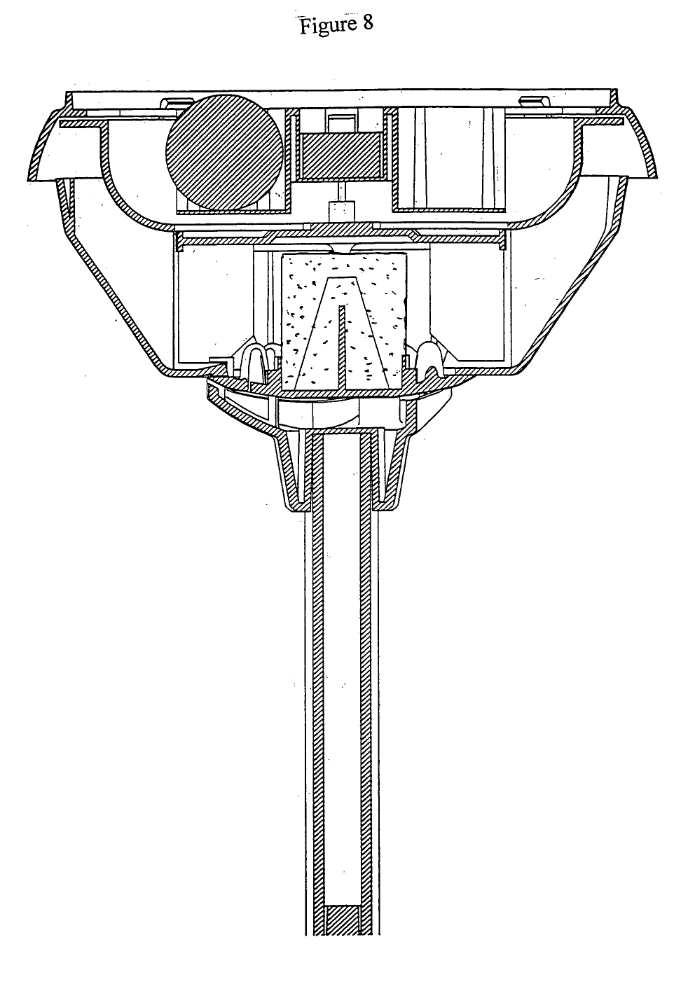

### VERSIOM 1

The 5 amp mains electricity (1) provides the main power for both the light (2) and the control unit (3) and impeller (4) or trigger motor (5). The control unit supports rechargeable back up batteries (6), which will run the air freshener unit (11) for up to 24 hours. The batteries are charged from the 5 amp power supply aided by a transformer (7).

The power supply can drive the impeller (4) or trigger motor (5) to distribute the scent. With tungsten and halogen lights (8 & 9) they create a thermal circulation (10) that can also be used to distribute the scent.

During the period when the light (2) is off the backup batteries (6) continue to operate the luminair airfreshener combined unit (11). However in many cases the lighting circuit is part of a continuous power feed at the ceiling point. The on/off switch is often a separate cable link. Therefore mains power (1) would be on tap.

The control unit (3) would be a PCB and the mains power supply (1) would run via it and the backup batteries (6).

The impeller (4) can be operated by electricity and thermal circulation (10) from the tungsten or halogen light (8 & 9).

The control unit (3) activates the trigger motor (5) to distribute the scent evenly throughout the room.

The scent may be emitted in different directions (12) from the Luminair airfreshener combined unit to produce a more even distribution of scent around the room.

The components of the Luminair airfreshener combined unit are held within a casing (13) with a transparent area for emitting light, holes from which the scent can be emitted into the room (12), holes for being attached to a wall or ceiling, and an opening for the mains supply cable to be fed into.

The scent source is perfume in a contained form that can be replaced by the user.

### VERSIOM 2

The main body (21) comprises an out wall (14) and an inner wall (15). The area between the outer wall (14) and inner wall (15) is the inner chamber (20). Within the inner chamber (20) are a series of radial dividing walls (19) compartmentalizing the inner chamber. Centrally located is a cartridge opening (16) that retains a fragrance cartridge (17). Adjacent to the fragrance cartridge (17) and within the perimeter defined by the maximum diameter of the impeller (4) is a series of inlets (18) through which the air is drawn by the impeller (4). The configuration of the inlet (18) openings encourages a rotating airflow and for the air to pass over the surface of the centrally located fragrance cartridge (17). The impeller (4) directs the air from the center laterally outwards into the inner chamber (20). The radial dividing walls (19) within the inner chamber (20) encourage a cyclonic airflow. At the outer periphery of the outer wall (14) and inner wall (15) is an opening (23) configured to redirect the cyclonic air flow projecting it laterally and rotational outward and away from the luminair airfreshener combined unit (11) mounting surface (22).

As a further embodiment of the invention the fragrance cartridge (17) incorporates about the periphery of the fragrance medium (25) a series of directional inlets (24) that initiate and encourage the air to flow cyclonically by the impeller (4) and the radial dividing wall (19) within the main body (21).

The cartridge (17) incorporates retaining interlock features (26) used to secure the cartridge (17) into the opening (16).

The main body (21) incorporates a locking means (27) which interfaces with the retaining interlock features (26) of the cartridge (17) to inhibit the removal of the cartridge other than by way of a secondary component or key herein described as key (28).

The key (28) provides a means of support for the cartridge (17) when installing the cartridge into the main body (21). Additionally the key (28) incorporates protruding features (29) which act upon locking means (27) such as to displace said locking means (27) so permitting engagement of the retaining interlocking features (26) resulting in a condition where the cartridge (17) cannot be removed without use of the key (28). To remove the cartridge (17) the operation using the key (28) is carried out in reverse.

Where the luminair airfreshener combined unit (11) is ceiling mounted the airflow would be projected rotationally downwards into the room.

The advantages are:
1. The light is often positioned in the middle of the room resulting in a more even distribution of the scent.
2. In most toilet areas there is a power supply for the light available to use.
3. There is no requirement to install power supply on walls often decorated with tiles.
4. The power supply is continuous unlike solar or batteries that need replacing.
5. The unit cost should be similar to a stand-alone unit as much of the construction is common to both a light and an air freshener.
6. It is very easy to install.
7. The air that enters the unit then is encouraged into a cyclonic or rotational motion until being exhausted back into the atmosphere with a cyclonic and conical and spiralling motion. In so doing maximising an even distribution of the fragrance.
Drawing A: An example of how the Luminair airfreshener combined unit parts may be when incorporating a tungsten/halogen ceiling light.
Drawing B: An example of how the Luminair airfreshener combined unit parts may be when incorporating a fluorescent light.
Drawing C: An example of how the luminair airfreshener combined units may be without a light source from a central section view.
Drawing D: An example of how the luminair airfreshener combined units may be without a light source from a top view
Drawing E: An example of the cartridge and key assembly prior to installation in the main body
Drawing F: A general sectional view through the complete assembly prior to installing the fragrance cartridge
Drawing G: A general isometric view
Drawing H: A section view of the assembled unit and the cartridge removal key.

1. A ceiling mounted air freshener comprising a means of distributing fragrance into the atmosphere in combination with a light source.

2. The air freshener of claim 1, further comprising an impeller, which is driven both by a motor and by thermal circulation caused by Wheat from the light source.

3. The air freshener of claim 1, wherein the air freshener can function solely as a means of distributing fragrance into the atmosphere by means of an impeller.

4. The air freshener of claim 1, further comprising a main body assembly made up of an outer wall and an inner wall creating an inner chamber.

5. The air freshener of claim 1, further comprising an inner chamber which is sub-divided into compartments by a series of radial walls designed to encourage airflow into a cyclonic or radial motion.

6. The air freshener of claim 1, further comprising a main body, which houses an impeller, wherein the impeller draws air into its center and directs the air laterally outward.

7. The air freshener of claim 6, further including a cartridge having a fragrance medium, the cartridge being centrally located within the main body and adjacent to the center of the impeller.

8. The air freshener of claim 7, wherein the cartridge incorporates a series of inlets radially about the perimeter of the cartridge such that the air being drawn in by the impeller can pass over the fragrance medium.

9. The air freshener of claim 8, wherein the inlets are shaped as to encourage the air into a cyclonic or rotational motion before entering the impeller.

10. The air freshener of claim 1, further comprising a main body which has at the outer periphery an opening configured to redirect the air flow projecting it laterally and rotationally outward and away from the mounting surface.

11. The air freshener of claim 1, wherein a configuration and combination of a main body assembly and the air freshener will draw air in through inlets, allow the air to pass over a cartridge, in so doing collect fragrance.

12. The air freshener of claim 1, wherein an airflow upon entering the air freshener then is encouraged into a cyclonic or rotatable motion until being exhausted back into the atmosphere with a cyclonic and conical and spiraling motion.

13. The air freshener of claim 12, further comprising a cartridge incorporating a series of directional inlets to direct the airflow.

14. The air freshener of claim 1, further comprising an interlocking interface means on a main body and a cartridge releasable only by a dedicated key which displaces a locking means on the main body permitting security of the cartridge.

15. (canceled)
